(19) United States
(12) Patent Application Publication
(10) Pub. No.: US 2025/0235878 A1
(43) Pub. Date: Jul. 24, 2025

(54) STABLE IONIC XANTHATE COMPOSITIONS IN AQUEOUS SOLUTION

(71) Applicant: OXIQUIM S.A., Santiago (CL)

(72) Inventor: Marcos Antonio MORALES HERRERA, Santiago (CL)

(21) Appl. No.: 18/853,988

(22) PCT Filed: Mar. 21, 2023
(86) PCT No.: PCT/CL2023/050024

§ 371 Date: Oct. 3, 2024

(30) Foreign Application Priority Data
Apr. 4, 2022 (CL) 850-2022

(51) Int. Cl. B03D 1/012; B03D 1/008

(52) U.S. Cl. CPC B03D 1/012; B03D 1/008; B03D 2201/02

(57) ABSTRACT

The present invention relates to stable ionic xanthate compositions in aqueous solution for prolonged periods of time that are useful as collector reagents in the froth flotation process. The stable ionic xanthates of the present invention are dissolved in aqueous solution at their maximum water solubility (28-30%), being highly stable and safe ionic products, preventing the instability and flammability of the solid products which allows safe handling, both for operators in mining work and for the environment.

### FIELD OF THE INVENTION

The present invention relates to aqueous compositions of xanthates stable in aqueous medium, such as ethyl (EXS), isopropyl (IPXS), isobutyl (IBXS) and amyl (PAX) xanthates, and salts thereof, and their method of preparation in order to be used directly in froth flotation processes of sulphide minerals without the need to carry out a previous treatment of the flotation reagent.

Xanthates of the present invention have the property of being ionic xanthates which have the advantage of being stable over time in spite of their liquid state at their maximum solubility concentration in water.

### BACKGROUND OF THE STATE OF THE ART

Xanthates are widely used in the mining industry in mineral flotation processes, and for decades different types of chemicals, including those mentioned above, IEXS, IPXS, IBXS, PAX and their derivatives have been used to collect valuable species at the lowest possible cost.

Xanthates are noble, low cost and stable molecules in a solid state that present a polar group containing bivalent sulfur of the R—O—CS₂ type, which is the one that interacts with the metal providing the hydrophobicity characteristics to the ore. Therefore, the functional group providing the hydrophobic characteristics is an anionic sulfhydryl group.

Xanthates are sodium or potassium salts of xanthogenic acid, which are stable in solid state and widely used because of their low cost, and high selectivity as collectors. Commercially, they are found in the form of yellow powders or crystals.

They are manufactured basically from 3 elements: carbon disulfide (CS₂), an alkali (sodium or potassium hydroxide), and an alcohol (methanol, ethanol, propanol, etc.). It is important to mention that they must be used in a neutral or alkaline medium, since in an acid medium they undergo hydrolysis and therefore lose their collecting properties.

State-of-the-art evidence indicates that xanthates in aqueous solutions and absence of minerals decompose easily producing carbon disulfide (CS₂). The generation of CS₂ is pH dependent, therefore, the decomposition of xanthates produces toxic waste that is dangerous for operators and harmful for the environment, since it generates a volatile, colourless and highly flammable liquid, which is a permanent concern in the mining industry. In addition, due to the partial or total hydrolysis of xanthates, hydrogen sulfide (H₂S) is released, which is also a highly toxic compound.

Currently, the main methods of xanthate production are:
Direct synthesis method: industrial production of xanthates is carried out in batches through the kneading method, where alcohol and carbon disulfide are added to a kneader, and then an alkali is added in a powder form. Production of xanthate by this kneading method is difficult to achieve since different variables must be controlled, for example it is required that the alkali has a fine particle size, and to achieve the desired particle size high energy consumption is required to pulverize the alkali. In addition to this high energy consumption, the xanthate synthesis reaction is highly exothermic and requires a high-power refrigerator to strictly control the temperature of the reaction, otherwise it not only affects the quality of the product, but can also lead to dangerous exothermic reactions. In summary, it is difficult to achieve batch production using this method, as there are also losses of volatile reagents, such as carbon disulfide, in the production process. Moreover, as temperature is a difficult parameter to control during the production process, it results in incomplete reactions, plus secondary reactions, resulting in a low purity product.
Wet alkali method: involves adding a small amount of water during the production of sodium alkoxide to moisten the caustic soda (sodium hydroxide), thus preventing agglomeration of the caustic soda and completing the reaction. The prepared sodium alkoxide is reacted with carbon disulfide to form xanthate (i.e., an aqueous xanthate solution). The liquid xanthate products obtained by this method have the advantages of a low cost of production, do not need to be dissolved when used, are easy to operate and have a controllable amount of free alkali in the product. This method is mainly suitable for small-scale local production. However, the liquid xanthate obtained by this method is unstable and difficult to store, which greatly limits its application, since once produced it must be used immediately.
Crystallization method: caustic soda and alcohol are converted into sodium alkoxide in a large amount of organic solvents such as benzene and gasoline, and then are reacted with carbon disulfide to crystallize the resulting xanthate in solution, they are filtered and then dried to obtain the product. The xanthate produced by this method is of good quality, but the production cost is high.

Although the solid-state xanthates described in the state of the art provide broad use, low cost and metallurgical virtues, they have two characteristics that are problematic for their use in industry and environmental impact:
1.—Xanthates are hazardous and highly flammable products in solid state due to the generation of CS₂, which forces the industry to comply with known standards for hazardous product handling. Therefore, given their high flammability, they require storage and distribution logistics that must comply with, among other things, confinement and construction of bunkers for storage, qualified personnel and certified facilities (anti-explosion) for their daily handling, which translates into high investments to minimize the risk together with continuous monitoring in this regard.
2.—Xanthates in flotation processes are used in aqueous solution at approximately 10% concentration. Batch production is carried out in quantities or volumes that are generally consumed in a day of operation, that is, in approximately 22 to 26 hours, because when xanthates are diluted in water they decompose into CS2, alcohol and carbonate salts and sulfurocarbonates, losing their metallurgical and collecting properties in 24 to 26 hours after the solution is produced.

CN107698475 discloses the synthesis of sulfide mineral collectors and, in particular, relates to a process of synthesizing liquid xanthate under organic solvent-free conditions with the objective of facilitating the recovery of xanthates at the end of the synthesis process and avoiding the difficult recovery of organic solvents. The process includes the steps of synthesizing liquid sodium butyl xanthate/potassium butyl xanthate under the condition that it is free of organic solvents, in a three-hole flask equipped with a thermometer, a stirrer, a dropping funnel, a condensing tube and a water bath. The process consists of adding butanol and a carbon disulfide solution, thoroughly stir the mixed solution and heat to raise the temperature to 28-35° C.; slowly adding a sodium hydroxide solution by drops into the mixed solution in the three-neck flask, controlling the drop addition rate to be 1-3 ml/min, while stirring; and upon completion of the dropwise addition of the sodium hydroxide solution, stirring and heat preservation to obtain liquid sodium butyl xanthate/potassium butyl xanthate. The method of preparation of the liquid xanthate is carried out at atmospheric pressure, with control of reaction temperature and the rate of dropwise addition of sodium hydroxide solution.

The liquid sodium butyl xanthate/potassium butyl xanthate obtained in this document is a product that must be used within a short period of time, since it is not stable for prolonged periods, so it cannot be stored. Therefore, this document does not aim to solve the technical problem of stability of xanthates in aqueous medium and stabilization for prolonged periods. Thereby, the product disclosed in document CN107698475 is not a product that can be stored and requires to be prepared at the time of its use.

Document CN107235879 also refers to the synthesis of flotation reagents, in particular to a method for synthesizing liquid xanthate. The compound is synthesized by mixing ethanol, CnH₂n+OH and carbon disulfide in a certain molar ratio, and then adding a certain proportion of solution of a strong alkali into the solution mixture. The synthesis method provided by this document adopts a reverse loading method, at first alcohol and carbon disulfide are mixed, and then an alkali liquor is added to the mixed solution, so as to obtain liquid xanthate. Although this document highlights that the synthesis of liquid xanthate has the advantages of being a simple production process, less investment in equipment, safe working environment, low labor intensity, and that it is not necessary to dry the product, among other advantages, it is not established as a technical problem to obtain a liquid xanthate in a stable aqueous medium for a prolonged period of time. The xanthate obtained in this document should be used within a short period of time.

JPS567757 describes the preparation of a liquid xanthate in an organic solvent, such as benzene, toluene, xylene, etc., crystallized in the solvent and extracted with water. The target liquid xanthate is separated directly from the solvent. The organic solvent can be recycled. The preparation steps can be reduced compared to the conventional process comprising separation of the crystallized xanthate and drying of crystals. The main goal in this document is to recover organic solvents with the least amount of impurities by a liquid-liquid separation of the xanthate solution. Therefore, the technical problem addressed in this document aims at the recovery of solvents and not at obtaining stable xanthates in aqueous medium for a long time, since the liquid xanthate obtained must be used in a short period of time.

Unlike what is described in the prior art, the process of the present invention addresses and develops the technology to dilute the xanthate to its maximum solubility in water (28-30%), obtaining ionic products of high stability and safety, solving the two most important variables of its use in the mining industry, these are to eliminate flammability and increase stability in aqueous solution, since xanthates in solid state (powder or pellets) are flammable, and when dissolved in aqueous media are very unstable, generating volatile compounds that lose their properties and effectiveness over time. Therefore, the technology developed in the present invention provides a safe product with proven high stability in aqueous solution for at least 10 months, maintaining its metallurgical properties.

Therefore, the aqueous ionic xanthate technology of the present invention solves and eliminates flammability, providing high stability over time for safe and reliable handling, while maintaining efficiency and metallurgical performance.

Currently known industrial evidence indicates that the Australian company Coogee manufactures an aqueous liquid ethyl xanthate that is refrigerated and stored at 3-5° C. and with this it reaches a stability of 15 to 30 days, to maintain an adequate performance at the time of its use in the flotation process in the local mining industry. Unlike this commercial product, the ionic xanthate in aqueous solution of the present invention does not require refrigeration for storage and its proven shelf life can reach up to 10 months.

The main differences that make the technology of the present invention have considerable advantages to what is described in the state of the art and/or has been published or reported, are provided by its characteristics of null flammability and high stability over time, which makes it a product attractive in price that can be transported or exported worldwide with storage logistics that do not need to consider the handling of flammable products or the construction of bunkers under different environmental conditions without losing its effectiveness. Among its advantages it should also be considered production and storage methods with low production and operational costs, which make this technology highly competitive and with an excellent metallurgical performance.

### BRIEF DESCRIPTION OF THE DRAWINGS

FIG. 1. FT-IR spectra of a) sodium isopropylxanthate as a commercial reagent and b) stable ionic isopropylxanthate in aqueous medium.

FIG. 2. Reverse-phase HPLC chromatograms of (a) commercial sodium isopropylxanthate; (b) stable ionic isopropylxanthate in aqueous medium of the present invention. Chromatograms c) and d) correspond to magnifications of chromatograms a) and b) respectively.

Experimental conditions: Mobile phase (70% formic acid solution 0.1% and 70% acetonitrile, stationary phase (C-18, 25 cm), UV-VIS detector (301 nm)).

FIG. 3. FT-IR spectra and detailed chromatogram of stable ionic isopropylxanthate in aqueous medium according to the present invention a) and c) prepared at time 0; b) and d) 10 months after preparation.

FIG. 4. FT-IR spectra of a) sodium isobutylxanthate as a commercial reagent and b) stable ionic isobutylxanthate in aqueous medium.

FIG. 5. Reverse-phase HPLC chromatograms of (a) commercial sodium isobutylxanthate; (b) ionically stable isobutylxanthate in aqueous medium of the present invention. Chromatograms c) and d) correspond to the magnifications of chromatograms a) and b) respectively.

Experimental conditions: Mobile phase (70% formic acid solution at 0.1% and 70% acetonitrile, stationary phase (C-18, 25 cm), UV-VIS detector (301 nm)).

FIG. 6. FT-IR spectra and detail chromatograms of stable ionic isobutylxanthate in aqueous medium according to the present invention a) and c) prepared at time 0; b) and d) 10 months after preparation.

FIG. 7. FT-IR spectra of a) potassium amyl xanthate as a commercial reagent and b) stable ionic amyl xanthate in aqueous medium.

FIG. 8. Reverse-phase HPLC chromatograms of (a) potassium amyl xanthate (b) stable ionic amyl xanthate in aqueous medium of the present invention. Chromatograms c) and d) correspond to the magnifications of chromatograms a) and b), respectively.

Experimental conditions: Mobile phase (70% formic acid solution at 0.1% and 70% acetonitrile, stationary phase (C-18, 25 cm), UV-VIS detector (301 nm)).

FIG. 9. FT-IR spectra and detail chromatograms of the stable ionic amyl amyl xanthate in aqueous medium according to the present invention a) and c) prepared at time 0; b) and d) 10 months after preparation.

FIG. 10. Metallurgical behavior over time of stable ionic liquid isopropylxanthate (X30) in different ores versus a standard isopropylxanthate collector (IPXS). a) copper recovery percentage; b) iron recovery percentage; c) molybdenum recovery percentage.

### DETAILED DESCRIPTION OF THE INVENTION

The present invention is directed to provide stable xanthates in aqueous solution and its preparation method as collectors in the flotation stage in the mining industry presenting the advantage of being stable for a prolonged period of time, which allows its storage, transport, distribution and an easy and fast handling at industrial level in a safe way to obtain efficient results.

The inventors of the present invention have developed a method to maintain soluble and stable compounds derived from xanthates, by means of the formation of stable ionic xanthate products in aqueous solution, overcoming the usual problems presented by xanthates. In industry it has been reported that the chemical stability of xanthates is of 12 hours after solubilization. After this period there is a decrease in the efficiency as collectors. On the other hand, the ionic xanthates in aqueous solution obtained and developed in the present invention completely avoid the risk of exothermic reactions in solid state which may cause ignition or explosions.

In this way, the products of the present invention allow storage for a long time, which simplifies their transport and their direct use in the mining industry, without the need of a solubilization treatment prior to using, avoiding toxicity problems produced by the emanation of toxic gases such as CS₂ and H₂S and also avoiding the highly exothermic reactions that characterize these products as highly flammable.

The collectors developed in the present invention allow storing and transporting the compounds derived from xanthates, being “ready for use”, which facilitates and reduces the operating costs in the flotation process of the mining industry since they are stable for long periods of time, allowing greater durability of the product, and do not entail the risks of solid xanthates, since they do not release volatile toxic compounds and there is no risk of inflammation.

The preparation of the xanthates obtained by the method of the present invention is carried out mainly by the following stages:
Stage 1: in a suitable reactor and depending on the batch size to be produced, load 65-72% water, preferably 68-69% in relation to the total weight, at neutral pH (pH 6.5-7.0) and adjust the temperature to 5-30° C., preferably between 15-25° C.
Stage 2: maintain an agitation of 15-100rpm, preferably between 30-70rpm, and add over the agitated water the xanthate in a concentration of 25-31%, preferably 28-30% in relation to total weight. The xanthate is added in solid form, either as granules or powder. The xanthates are chosen from ethyl (EXS), isopropyl (IPXS), isobutyl (IBXS) and/or amyl (PAX) xanthate.
Step 3: stir for 5-30 minutes, preferably 5-10 minutes, until total dissolution of the solid.
Stage 4: after total dissolution of the solid, add 0.5 to 5.0% by weight or preferably 1 to 3% relative to the total weight a nitrogenous base, selected from monoethanolamine, diethanolamine, triethanolamine, or mixtures thereof until reaching a pH of 10-12, and stir for 3-20 minutes, preferably between 3-7 minutes.
Stage 5: add under constant stirring between 1 to 5% by weight, preferably 2.3 to 3% relative to the total weight of a low molecular weight alcohol, selected from methanol, ethanol, propanol, isopropanol, butanol, isobutanol, secbutanol, n-pentanol, 2-pentanol, 3-pentanol, hexanol, amyl alcohol, preferably isopropanol and isobutanol. The reaction mixture is stirred for 3 to 30 minutes, preferably 3-7 minutes.

The products obtained by the method of the present invention result in ionic xanthates stable in aqueous medium.

Examples of preparation of aqueous compositions of ionic xanthates and their stability over time.

### 1. Preparation of a Stable Sodium Isopropylxanthate Composition in Aqueous Solution

In a glass or carbon steel reactor filled with 70 liters of water at neutral pH (pH 6.5-7.0), 28 kg of sodium isopropylxanthate in granules or powder form is added at a temperature of 25° C. It is kept under agitation for 30 minutes, until total dissolution of the solid. Subsequently, 1 kg of an ethanolamine/triethanolamine mixture (1:1) and 2.5 kg of isopropanolanol are added until a pH of 10-12 is obtained, and stirred for 20 minutes.

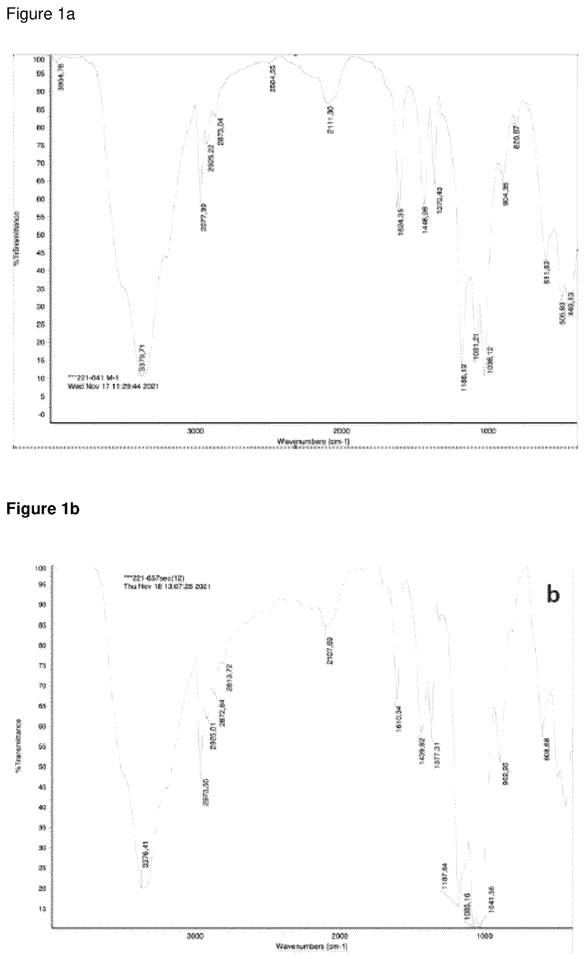

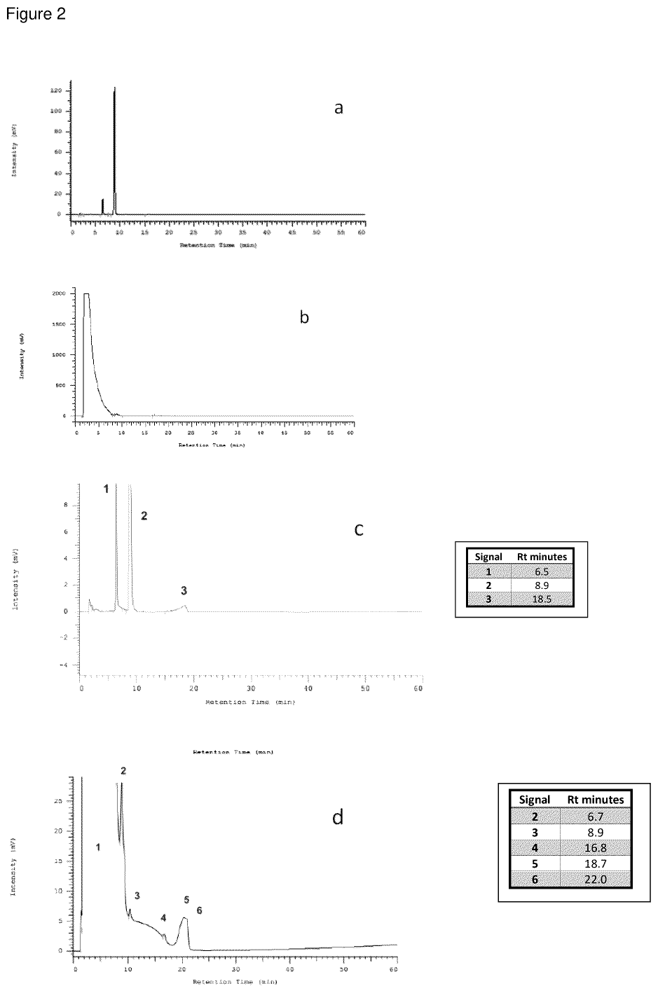

The reaction product results in a stable ionic isopropylxanthate as shown in FIGS. 1b, 2b and 2d.

FIG. 1a) shows the FT-IR spectrum of the commercial product sodium isopropylxanthate showing the C═S, C—O—C bond tension bands symmetric and asymmetric at 1036 cm⁻¹, 1091 cm⁻¹ and 1188 cm⁻¹, respectively. FIG. 1b) corresponds to the FT-IR spectrum of the reaction product of the present invention, it shows insignificant shifts of the bands attributed to the C═S and C—O—C bond tension of 6 and −6 cm⁻¹, respectively. This observation evidences that the R—O—CS₂ functional group is not altered, so it remains free to interact with a metal or metal ion.

FIGS. 2-a and 2-c show the reverse-phase HPLC chromatographic analysis of an aqueous solution of a commercial standard of sodium isopropylxanthate and FIGS. 2-b and 2-d) show an aqueous solution of the ionic isopropylxanthate reaction product of the present invention. As it is possible to observe from the chromatograms, a modification of the polarity of the sodium isopropylxanthate components (FIG. 2-a) with respect to the components of the stable ionic isopropylxanthate of the present invention (FIG. 2-b) stands out. The signal corresponding to isopropylxanthate (rt=6.5 minutes) decreases significantly its concentration in the product of the present invention (FIG. 2-d) and a signal assigned to an ionic compound appears below the 5 minutes retention time. Besides this major compound, nonpolar compounds appear in a lower proportion, which can be classified as by-products, corresponding to impurities of the initial product and which do not interfere with the metallurgical process (FIG. 2-d).

In conclusion, there is experimental evidence supporting that the stable isopropylxanthate reaction product stable in aqueous medium of the present invention is a stable ionic product, different from the commercial product, that keeps the R—O—CS₂ group free for interaction with a metal or metal ion to allow efficient flotation.

### Stability of Isopropyl Isopropylxanthate Composition in Aqueous Solution

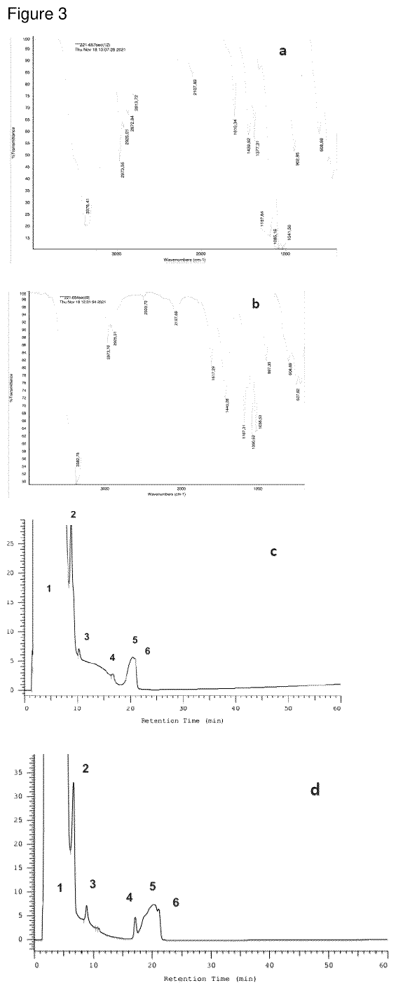

FIG. 3 shows the FT-IR spectra and details of the chromatograms of isopropylxanthate ionic product in aqueous solution of the present invention prepared at time 0 and 10 months after preparation. Experimental evidence is consistent with the stability of the isopropylxanthate ionic product. Chromatograms of the freshly prepared formulation and after 10 months showed no significant difference. The only appearance of a new minor by-product is observed between signals 4 and 5 in FIG. 3-d. In the FT-IR spectra, no significant shift of the bands corresponding to the R—O—CS₂ groups is observed. However, a relative increase of the band corresponding to the —OH stress band (3380 cm⁻¹) is observed which is consistent with the partial hydrolysis of the ionic product derived from the isopropylxanthate.

### 2. Preparation of a Stable Sodium Isobutylxanthate Composition in Aqueous Solution

In a glass or carbon steel reactor filled with 70 liters of water at neutral pH (pH 6.5-7.0), 28 kg of sodium isobutylxanthate in granules are added at a temperature of 25° C. It is kept under agitation for 30 minutes, until total dissolution of the solid. Subsequently, 1 kg of a mixture of monoethanolamine/triethanolamine (2:1) and 2.5 kg of isobutanol is added until reaching a pH of 10-12, and stirred for 20 minutes.

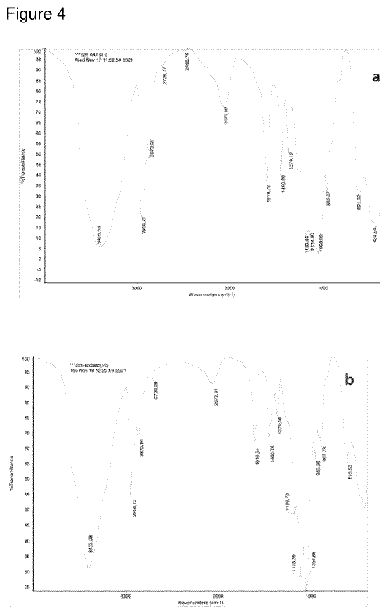

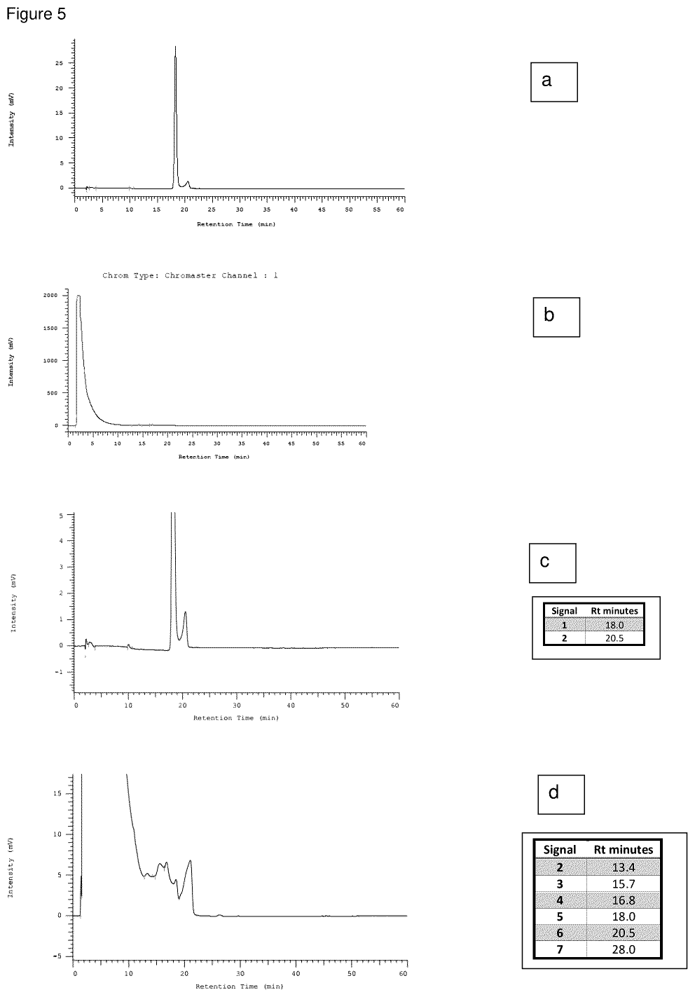

The reaction product results in a stable aqueous solution of ionic isobutylxanthate as shown in FIGS. 4b, 5b and 5d.

FIG. 4-a shows an FT-IR spectrum of the commercial sodium isobutylxanthate product showing the C═S, C—O—C bond tension symmetric and asymmetric bands at 1059 cm⁻¹, 1114 cm⁻¹ and 1189 cm⁻¹, respectively. FIG. 4-b shows the FT-IR spectrum of the ionic isobutylxanthate product of the present invention where no significant shifts of the bands attributed to the C═S and C—O—C bond tension were observed, which evidences that there is no alteration of the R—O—CS₂ group in the compound of the present invention, in the same way as was observed for the isopropylxanthate described previously, so that it remains free to interact with a metal or metallic ion.

FIGS. 5-a and 5-c show the reverse phase HPLC analysis of a commercial standard of sodium isobutylxanthate in an aqueous solution and FIGS. 5-b and 5-d show the reaction product of the present invention, an aqueous solution of ionic isobutylxanthate. As it can be seen from chromatographic analysis (FIG. 5), a significant modification of the polarity of sodium isobutylxanthate (FIG. 5-a) with respect to the ionic isobutylxanthate of the present invention (FIG. 5-b) is shown, similar to what is observed in FIG. 2 for isopropylxanthate. Modification of the retention time of most of the components towards retention times shorter than 5 minutes indicates the formation of the ionic compound, which retains the free R—O—CS₂ group according to the information provided by the IR spectrum in FIG. 4.

In the magnified chromatogram of ionic isobutylxanthate of the present invention (FIG. 5-d) the original signals of isobutylxanthate (rt=18 and 20.5 minutes) that did not react and other minor by-products of different polarity are observed.

### Stability of Ionic Isobutylxanthate Composition in Aqueous Solution

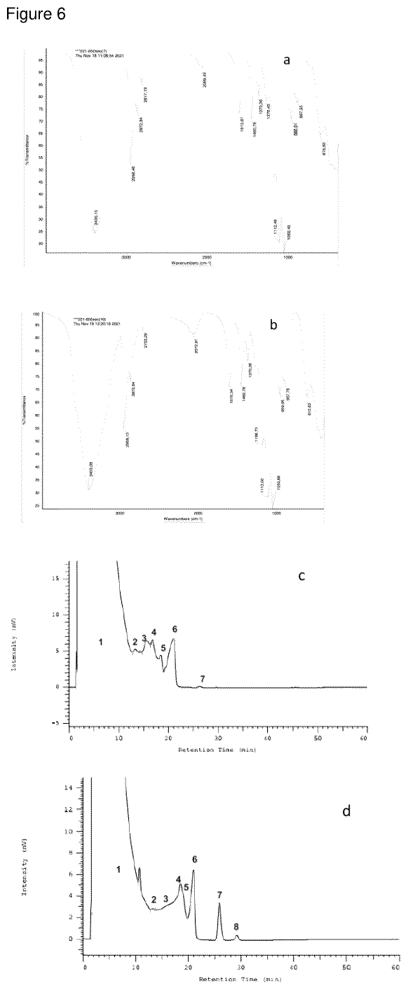

FIG. 6 shows the FT-IR spectra and details of the chromatograms of stable aqueous ionic isobutylxanthate composition of present invention prepared at time 0 and 10 months after preparation. Experimental evidence is consistent with the stability of the ionic isopropylxanthate derivative product.

No shift of the bands corresponding to the R—O—CS₂ groups is observed in the FT-IR spectra, and no relative changes in the intensity of bands is observed.

Chromatograms of the ionic isobutylxanthate aqueous solution of the present invention, at time 0 and after 10 months, showed no difference in the signal corresponding to ionic compounds. However, changes are observed in the minor nonpolar compounds (FIG. 6-d). It is highlighted the decrease in signal of isobutylxanthate (signal 5, FIG. 6-c and 6-d), the increase of a nonpolar by-product (signal 7) and the appearance of a new nonpolar product (signal 8).

These results are consistent with the stability of major ionic compounds and the transformation of minor compounds.

### 3. Preparation of a Stable Potassium Amyl Xanthate Composition in Aqueous Solution

In a glass or carbon steel reactor filled with 70 liters of water at neutral pH (pH 6.5-7.0), 28 kg of potassium amyl xanthate granules are added at a temperature of 25° C. It is kept under agitation for 30 minutes, until total dissolution of the solid. Subsequently, 1 kg of a mixture of monoethanolamine/triethanolamine (2:1) and 2.5 kg of amyl alcohol is added until a pH of 10-12 is obtained, and stirred for 20 minutes.

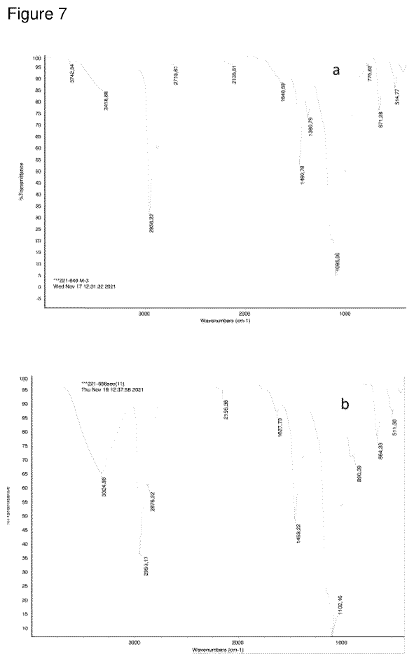

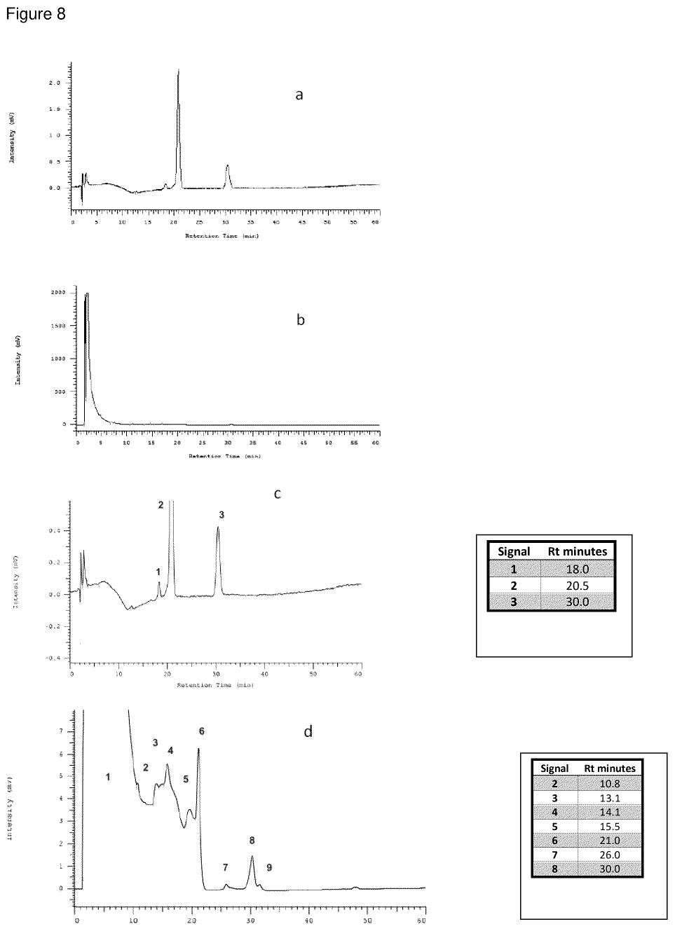

The reaction product results in an aqueous solution of stable ionic amyl xanthate as shown in FIGS. 7b, 8b and 8d.

FIG. 7 shows an FT-IR spectrum of commercial potassium amyl xanthate product (a), and the reaction product of an aqueous solution of stable ionic amyl xanthate of the present invention from (b), which exhibit C═S, C—O—C symmetric and asymmetric bond stress bands at 1010 cm⁻¹, 1100 cm⁻¹ and 1150 cm⁻¹, respectively. No significant differences are observed between commercial potassium amyl xanthate product and the stable ionic amyl xanthate reaction product of the present invention regarding relative position and intensity of the bands. Therefore, in the same way as mentioned for the previously analyzed compounds, no changes in the R—O—CS₂ functional group are evidenced, which shows that there is no alteration of the R—O—CS₂ group in the compound of the present invention, in the same way as it was observed for the isopropyl xanthate and isobutyl xanthate described previously, so the bond remains free to interact with a metal or metallic ion.

The chromatograms shown in FIG. 8, display a significant modification of the polarity of the aqueous compositions of ionic xanthates in a similar way to the observed for the ionic xanthates in aqueous solution analyzed previously.

The modification of the retention time of most of the components towards retention times shorter than 5 minutes indicate the formation of ionic compounds which retain the free R—O—CS₂ group according to the information provided by the IR spectra in FIG. 7. In the magnified chromatogram of the formulation (FIG. 7-d), the original unreacted amyl xanthate signals (tr=21.0 and 30 minutes) are observed. Among the minor compounds and unlike the previous samples, FIG. 7-d shows the appearance of at least 4 compounds that are more polar than commercial amyl xanthate but are not ionic.

### Stability of the Amyl Ionic Xanthate Composition in Aqueous Solution

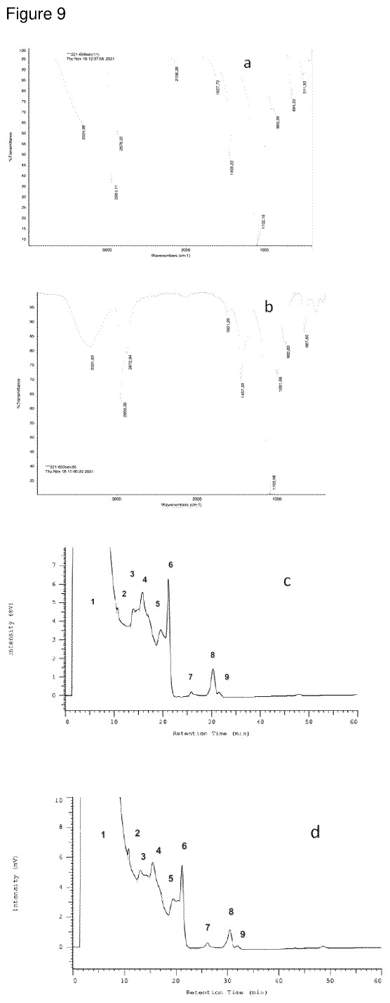

FIG. 9 shows the FT-IR spectra and details of the chromatograms of the stable aqueous amyl ionic xanthate composition of the present invention prepared at time 0; and 10 months after preparation. The experimental evidence is consistent with the stability of the ionic amyl xanthate derivative product.

In the FT-IR spectra no shift of the bands corresponding to the R—O—CS₂ groups is observed, and no relative changes in the intensity of the bands is observed.

Chromatograms of the ionic amyl xanthate of the present invention, at time 0 and after 10 months, did not show differences in the signal corresponding to the ionic compounds nor in minor components (FIG. 9-c and 9-d).

### Recovery of Metals Method by Means of the Aqueous Xanthate Compositions of the Present Invention

To evaluate the metallurgical performance, a flotation process was carried out using as collector the stable ionic xanthate compositions in aqueous solution of the present invention. The compositions of the invention were tested and the flotation process carried out with the stable ionic isobutyl xanthate composition in aqueous solution obtained by the process described in the present invention is depicted below.

The tests were designed at scale with a validated evaluation system in a simulated environment using laboratory metallurgical flotation cells with a Rougher flotation equipment. Recovery of a number of metals was tested by trying different ores.

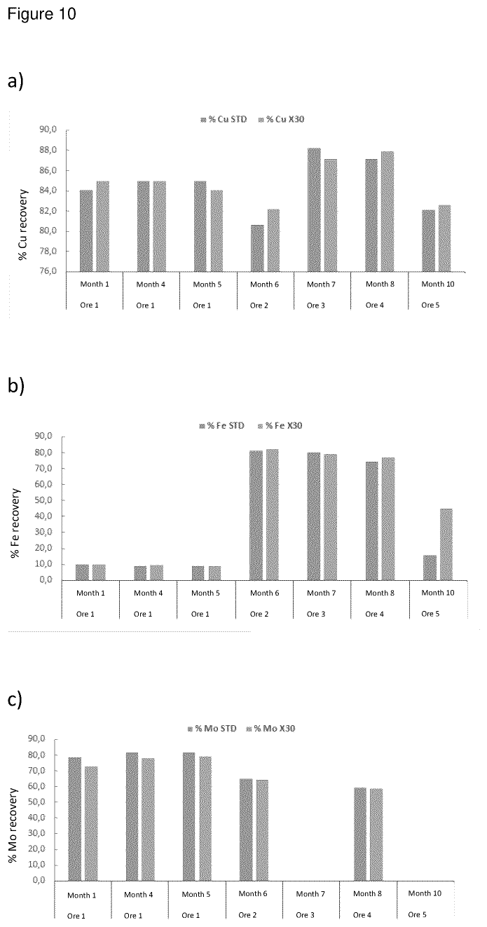

FIG. 10 was constructed based on Table N° 1 included below and shows the metallurgical behavior over time of the aqueous stable ionic isopropylxanthate composition of the present invention prepared at time 0, and used as a collector at times 1, 4, 5, 6, 7, 8 and 10 months, as compared to a standard solid isopropylxanthate which was prepared at the time of use. To measure the efficiency of the aqueous solution stable ionic isopropylxanthate composition of the present invention, recovery of copper, iron and molybdenum was measured and compared to the standard method consisting of using solid xanthates dissolved in aqueous medium at the time of use.

TABLE No1
Metallurgical results of aqueous compositions of stable ionic isopropylxanthate
of the present invention compared to a standard isopropylxanthate solution.

| | | Cu | Fe | Mo | Aqueous | | | | | | |
|---|---|---|---|---|---|---|---|---|---|---|---|
| | | grade | grade | grade | Xanthate | % | % | % | % | % | % |
| Month | Ore | % | % | (ppm) | Stability | Cu_{std} | Cuₓ₃₀ | Fe_{std} | Feₓ₃₀ | Mo_{std} | Moₓ₃₀ |
| 1 | Ore 1 | 0.337 | 4.85 | 130 | Month 1 | 84.08 | 85.00 | 10.00 | 10.01 | 78.88 | 73.02 |
| 4 | Ore 1 | 0.337 | 4.85 | 130 | Month 4 | 85.00 | 84.99 | 9.21 | 9.81 | 81.91 | 78.00 |
| 5 | Ore 1 | 0.337 | 4.85 | 130 | Month 5 | 85.00 | 84.09 | 9.21 | 8.81 | 81.91 | 79.08 |
| 6 | Ore 2 | 1.19 | 3.58 | 242 | Month 6 | 80.70 | 82.20 | 81.10 | 82.40 | 65.30 | 64.80 |
| 7 | Ore 3 | 0.417 | 3.31 | | Month 7 | 88.26 | 87.21 | 80.10 | 78.95 | | |
| 8 | Ore 4 | 0.494 | 2.63 | 39 | Month 8 | 87.18 | 87.88 | 74.51 | 77.07 | 59.07 | 59.00 |
| 10 | Ore 5 | 0.471 | 1.9 | | Month 10 | 82.11 | 82.66 | 15.98 | 45.22 | | |

For this study, 7 independent flotation processes were considered, with ores 1, 2, 3, 4 and 5 comprising different copper, iron and molybdenum grades being tested as indicated in Table N° 1.

The goal of this study was to demonstrate that the xanthate compositions in aqueous solution of the present invention are as efficient as a standard xanthate prepared at the time of its use when used as collectors in a flotation process.

Each ore was subjected to an independent flotation and mineral extraction procedure, in order to demonstrate the behavior of the aqueous solutions of ionic xanthate of the present invention stored for up to 10 months and used at different times, as compared to a standard xanthate that was prepared moments prior to use.

There are no significant differences in the recovery of copper, iron and molybdenum, thus demonstrating that the compositions of ionic xanthates in aqueous solution, stored for up to 10 months, are as efficient as a standard solid xanthate prepared prior to its use in the mine site. In addition, it is observed that the recovery efficiency is independent of the treated ore.

Therefore, the advantages provided by the aqueous compositions of stable ionic xanthates of the present invention, allow it to be used directly in the froth flotation process without the need to perform a previous treatment of the flotation reagent, unlike what occurs with standard xanthates which require to be dissolved prior to their use. On the other hand, the xanthates in aqueous solution of the present invention do not decompose producing carbon disulfide (CS₂) or other toxic residues dangerous for operators and harmful for the environment, they do not hydrolyze so hydrogen sulfide (H₂S) is not released and they can be stored without risk of inflammation or explosion, favoring their use in the mining industry, besides reaching metal recoveries equivalent to the xanthate collector products commonly used in mining and already known in the state of the art.

1. An aqueous composition useful as a collector reagent in the froth flotation process wherein the composition is a stable aqueous solution comprising a ionic xanthate, a nitrogenous base and a low molecular weight C2-C6 alcohol.

2. An aqueous composition according to claim 1 wherein the xanthates are selected from sodium isopropylxanthate, sodium isobutylxanthate, potassium amyl xanthate.

3. An aqueous composition according to claim 1 wherein the nitrogenous base is selected from monoethanolamine, diethanolamine and triethanolamine and mixtures thereof.

4. An aqueous composition according to claim 1 wherein the alcohol is selected from methanol, ethanol, propanol, isopropanol, butanol, isobutanol, secbutanol, n-pentanol, 2-pentanol, 3-pentanol, hexanol, amyl alcohol.

5. An aqueous composition according to claim 4 wherein the alcohol is selected from isopropanol, isobutanol, amyl alcohol.

6. A method of preparation of the aqueous composition useful as a collector reagent in the froth flotation process that comprises the stages of:
a) loading a reactor with 65-72% of water in relation to the total weight, at neutral pH, and at a temperature of 5-30° C., preferably between 15-25° C.
b) maintaining constant agitation of 15-100 rpm, preferably between 30-70 rpm, and adding on the water in agitation the solid xanthate in granules or powder in a concentration of 25-31%, preferably 28-30% in relation to the total weight;
c) maintaining in constant agitation during 5-30 minutes, preferably between 5-10 minutes, until total dissolution of the solid.
d) adding a nitrogenous base in a concentration of 0.5 to 5.0% by weight or preferably 1 to 3% in relation to the total weight, until a pH of 10-12 is obtained, and stirring for 3-20 minutes, preferably between 3-7 minutes.
e) adding an alcohol of low molecular weight between 1 to 5% by weight, preferably 2.3 to 3% in relation to the total weight, under constant stirring.

7. A method according to claim 6 wherein the xanthates are selected from sodium isopropylxanthate, sodium isobutylxanthate and potassium amyl xanthate.

8. A method according to claim 6 wherein the nitrogenous base is selected from monoethanolamine, diethanolamine and triethanolamine or mixtures thereof.

9. A method according to claim 6 wherein the alcohol is selected from methanol, ethanol, propanol, isopropanol, butanol, isobutanol, secbutanol, n-pentanol, 2-pentanol, 3-pentanol, hexanol and amyl alcohol.

10. A method according to claim 9 wherein the alcohol is preferably selected from isopropanol, isobutanol and amyl alcohol.

11. A method of metal recovery by froth flotation wherein an aqueous composition of stable ionic xanthates according to claim 1 is used as collector.
